(19) United States
(12) Patent Application Publication
(10) Pub. No.: US 2025/0017313 A1
(43) Pub. Date: Jan. 16, 2025

(54) SYSTEMS AND METHODS FOR TESTING AND CHARACTERIZING ERGONOMIC PERFORMANCE OF FLOORING

(71) Applicant: SHAW INDUSTRIES GROUP, INC., Dalton, GA (US)

(72) Inventors: Barrett Randolph Morton, Cartersville, GA (US); Julie Brumbelow, Hilton Head, SC (US); Mattie Beavers, Chatsworth, GA (US); Samuel Huzzard, Northhamptonshire (GB); Tom Bayes, Northhamptonshire (GB); Zach Armitage, Northhamptonshire (GB)

(21) Appl. No.: 18/834,884

(22) PCT Filed: Jan. 31, 2023
(86) PCT No.: PCT/US2023/011985

§ 371 Date: Jul. 31, 2024

Related U.S. Application Data
(60) Provisional application No. 63/305,068, filed on Jan. 31, 2022.

(51) Int. Cl. A43B 3/44; G01M 99/00

(52) U.S. Cl. CPC A43B 3/44; G01M 99/007

(57) ABSTRACT

A method can comprise applying a force to a first sample, the force being representative of a bodyweight load, the first sample comprising a portion of a footwear article. One or more sensors can generate at least one first output in response to application of the force to the first sample. The force can be applied to a second sample, the second sample comprising a flooring material positioned beneath the portion of the footwear article in a stacked arrangement. The one or more sensors can generate at least one second output in response to application of the force to the second sample. Based on the at least one first output and said at least one second output, at least one value comparing the at least one first output to the at least one second output.

### CROSS-REFERENCE TO RELATED APPLICATIONS

This application claims priority to and the benefit of the filing date of U.S. Provisional Application No. 63/305,068, filed Jan. 31, 2022, the entirety of which is hereby incorporated by reference herein.

### FIELD

This application relates generally to apparatuses and systems for testing and characterizing flooring materials and, in particular, to evaluating and relating ergonomic performance of the flooring materials.

### BACKGROUND

Conventionally, the comfort of flooring materials have been presented to consumers using measurement values of raw physical characteristics, such as, for example, compression force, recovery, impact absorption, and shock absorption. Such raw physical characteristics are difficult for a lay consumer to comprehend and relate to. Thus, there is a need for relating flooring material comfort to the lay consumer in familiar terms. A way to relatably convey subjective flooring material properties such as comfort is desirable.

### SUMMARY

Described herein, in various aspects, is a method comprising applying a force to a first sample, the force being representative of a bodyweight load, the first sample comprising a portion of a footwear article. At least one first output can be generated, by at least one sensor, in response to application of the force to the first sample, wherein the at least one first output is associated with a first comfort indication metric. The force can be applied to a second sample, the second sample comprising a surface covering material (e.g., a flooring material) positioned beneath the portion of the footwear article in a stacked arrangement. At least one second output in response can be generated, by at least one sensor, to application of the force to the second sample, wherein the at least one second output is associated with a second comfort indication metric. Based at least in part on the first comfort indication metric and the second comfort indication metric, at least one value comparing the first comfort indication metric to the second comfort indication metric can be determined.

In another aspect, a method can comprise applying a force to a stacked arrangement of a flooring material and a portion of a footwear article, the force being indicative of a bodyweight load, the flooring material being positioned beneath the portion of the footwear article. At least one output can be generated, by at least one sensor, in response to application of the force to the stacked arrangement, wherein the at least one output is indicative of energy absorbed by the stacked arrangement. A shoe type that corresponds to the footwear article can be determined by a computing device in communication with a database comprising energy absorption properties of a plurality of shoe types. A relative increase in energy absorption provided by the flooring material in comparison to the shoe type can be determined by the computing device.

A system can comprise a force application assembly having a contact structure, an actuator coupled to the contact structure and configured to effect movement of the contact structure along a first axis, and at least one sensor. At least one processor can be in communication with the at least one sensor. Memory can be in communication with the at least one processor. The memory can comprise instructions that, when executed by the at least one processor, cause the at least one processor to: receive at least one first output from the at least one sensor in response to application of force to a first sample by the force application assembly, wherein the first sample comprises at least a portion of a footwear article; receive at least one second output from the at least one sensor in response to application of force to a second sample by the force application assembly, wherein the second sample comprises a flooring material and the portion of a footwear article provided in a stacked configuration in which the flooring material is beneath the footwear article; and determine, based on said at least one first output and said at least one second output, at least one value comparing the at least one first output to the at least one second output.

A system can comprise a force application assembly having: a contact structure, an actuator coupled to the contact structure and configured to effect movement of the contact structure along a first axis, and at least one sensor. At least one processor can be in communication with the at least one sensor. Memory can be in communication with the at least one processor. The memory can comprise a database comprising energy absorption properties of a plurality of shoe types. The memory can comprise instructions that, when executed by the at least one processor, cause the at least one processor to: receive at least one output from the at least one sensor in response to application of force to a stacked arrangement of a flooring material and at least a portion of a footwear article, wherein the at least one output is indicative of energy absorbed by the stacked arrangement; determine a shoe type that corresponds to the footwear article; and determine a relative increase in energy absorption provided by the flooring material in comparison to the shoe type.

In another aspect, a method can comprise applying a force to a first sample, the force being representative of a bodyweight load, the first sample comprising a portion of a footwear article. At least one first output can be generated, by at least one sensor, in response to application of the force to the first sample. The at least one first output can be associated with a first comfort indication metric. The force can be applied to a second sample, the second sample comprising a flooring material positioned beneath the portion of the footwear article in a stacked arrangement. At least one second output can be generated, by at least one sensor, in response to application of the force to the second sample. The at least one second output can be associated with a second comfort indication metric. The first comfort indication metric can be associated with the second comfort indication metric.

Additional advantages of the invention will be set forth in part in the description that follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

These and other features of the preferred embodiments of the invention will become more apparent in the detailed description in which reference is made to the appended drawings wherein:

FIG. 1 is a schematic diagram of an example system as disclosed herein with a first sample comprising a footwear article.

FIG. 2 is a schematic diagram of the example system of FIG. 1 with a second sample comprising the footwear article and a flooring material.

FIG. 3A is a perspective view of a portion of the example system in accordance with embodiments disclosed herein. FIG. 3B is a perspective view of a portion of another example system in accordance with embodiments disclosed herein.

FIG. 4A is a block diagram of an example operating environment of a flooring material comfort characterization device for use with the example system as disclosed herein.

FIG. 4B is a block diagram of an example operating environment of a flooring material comfort characterization device. FIG. 4C is a block diagram of a comfort engine. FIG. 4D is a block diagram of an example operating environment of a flooring material comfort characterization device.

FIG. 5 shows an exemplary chart comparing comfort indication metrics of exemplary flooring samples to comfort indication metrics of different shoe types.

FIG. 6 shows an exemplary chart comparing comfort indication metrics of exemplary flooring samples to comfort indication metrics of different shoe types. As shown, the comfort indication metrics are deceleration values that indicate energy absorption of the shoe type or flooring sample.

FIG. 7 illustrates an exemplary output showing pressure mapping distribution of an example test apparatus comprising an exemplary contact structure on different samples surfaces.

FIG. 8 illustrates another exemplary output showing pressure mapping distribution of an example test apparatus comprising another exemplary contact structure on different samples surfaces.

### DETAILED DESCRIPTION

The disclosed system and method may be understood more readily by reference to the following detailed description of particular embodiments and the examples included therein and to the Figures and their previous and following description.

It is to be understood that the terminology used herein is for the purpose of describing particular embodiments only and is not intended to limit the scope of the present invention which will be limited only by the appended claims.

It must be noted that as used herein and in the appended claims, the singular forms “a,” “an,” and “the” include plural references unless the context clearly dictates otherwise. Thus, for example, reference to “a sensor” includes one or more of such sensors, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Optionally, in some aspects, when values are approximated by use of the antecedents “about,” “substantially,” or “generally,” it is contemplated that values within up to 15%, up to 10%, up to 5%, or up to 1% (above or below) of the particularly stated value or characteristic can be included within the scope of those aspects.

The word “or” as used herein can mean any one member of a particular list and, unless context dictates otherwise, can also include any combination of members of that list.

Unless defined otherwise, all technical and scientific terms used herein have the same meanings as commonly understood by one of skill in the art to which the disclosed apparatus, system, and method belong. Although any apparatus, systems, and methods and materials similar or equivalent to those described herein can be used in the practice or testing of the present apparatus, system, and method, the particularly useful methods, devices, systems, and materials are as described.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

A technical problem that is solved by the systems and methods disclosed herein is a disconnect between measurement values taken during material testing of flooring materials and relation of said measurement values to a lay consumer in a meaningful and relatable way. A technical solution to said technical problem includes (1) obtaining a measurement value (e.g., an output from a measurement device or a comfort indication metric as further disclosed herein) of a flooring material and a footwear article; and (2) comparing the measurement value to another measurement value of the same and/or a different footwear article (or of a different flooring material or other structure providing a relatable amount of comfort, such as, for example, a yoga mat or standing desk mat). Accordingly, measurement values are consistent with (or representative of) a user experience of standing (or walking, etc.) on a particular flooring material while wearing a footwear article, which can be how the flooring material is most likely experienced during actual use. Moreover, the user experience can be related to other user experiences with which the user is familiar (e.g., standing in different shoe types that provide various degrees of comfort). Thus, the present disclosure further provides practical application of acquiring measurements of, and characterizing user experience of, flooring materials in ways that are consistent with actual user experience. Accordingly, the present disclosure is directed to the practical application of conveying user experience.

As used herein, a “comfort indication metric” can be defined as a measured value that is indicative of a comfort provided by a surface, such as, for example, an energy absorbed, a maximum deceleration or force value measured in response to an impact, a maximum pressure, an average pressure, or any other similar and appropriate measured value. In some optional aspects, the comfort indication metric can be a positive value. In further optional aspects, the comfort indication metric can be a negative value. As used herein, a “comfort improvement” can be a difference between a first comfort indication metric acquired from a first sample and a second comfort indication metric acquired from a second sample. For example, a first sample can provide a comfort indication metric of 10 units and a second sample can provide a comfort indication metric of 12 units, with the comfort indication metrics being determined in accordance with a common scale. In said example, the comfort improvement can be 2 units. As used herein a “relative comfort improvement” can be a ratio of a comfort improvement (provided by a flooring material) to a first comfort indication metric. Thus, for example, the relative comfort improvement of the example in this paragraph is 2/10 or 0.2 (20%). As used herein, a “relative comfort” can be a ratio of the first comfort indication metric to the second comfort indication metric. Thus, for example, the relative comfort of the example in this paragraph is 12/10 or 1.2. As used herein, a “sample” can be one or more materials that are tested with a test apparatus as disclosed herein to obtain a comfort indication metric associated with the one or more materials. As used herein, a “footwear article” can be any article that is worn on a foot of an individual, such as, for example, a boot, a dress shoe, a flip-flop, a running shoe, a nurse's shoe, or a high heel shoe. As used herein, a “flooring material” can be any material or combination of materials that can be used to form a surface upon which an individual can stand (e.g., optionally, hardwood, rubberized track, synthetic turf, carpet, carpet with underlay, composite or resilient floor, resilient with underlayment, laminate floor, etc.).

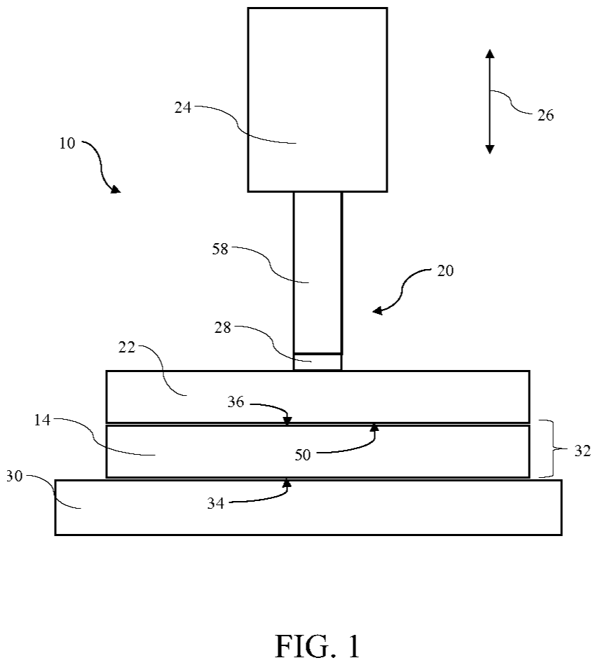

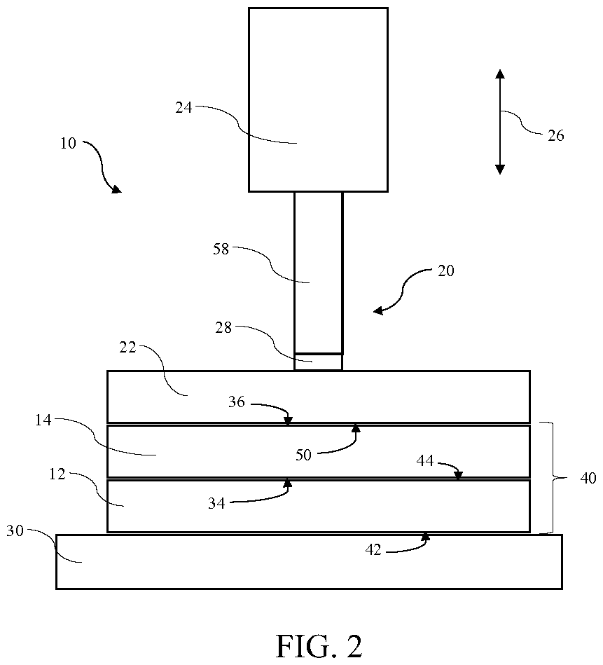

Referring to FIGS. 1-3A, B, an example system 10 can be used to acquire performance data of a flooring material 12 for relating said performance data of the flooring material to performance data of a footwear article 14. As can be understood, lay consumers are typically well associated with footwear, and can relate to and perceive the comfort of a footwear. As such, a lay consumer may be able to relate to the comfort of a flooring material when said comfort of the flooring material is compared to or against the more familiar comfort of footwear (e.g., a shoe or shoe type). Accordingly relation of the flooring material to footwear can provide a relevant comparison that a lay consumer can comprehend. It is contemplated that the footwear article 14 that is selected for comparison to the flooring material can be somewhat close to the comfort level of the floor in order to provide the most meaningful comparison.

The system 10 can comprise a force application assembly 20 having a contact structure 22. An actuator 24, such as, for example, a pneumatic or hydraulic cylinder, can be coupled to the contact structure 22 and can be configured to effect movement of the contact structure along a first axis 26 to apply a force to a sample placed below the contact structure 22. One or more sensors 28 can be associated with the force application assembly 20. The one or more sensors 28 can be configured to provide respective outputs in response to the force applied to the sample by the contact structure 22. The respective outputs provided by the one or more sensors 28 can be, or be associated with, measurements indicative of comfort provided by samples, i.e., comfort indication metrics. Location of the sensors shown in FIG. 1 is an example. In other embodiments, they can be located in any other appropriate position or location. For example, one or more of the sensors 28 can be placed in an anvil 30 below the samples.

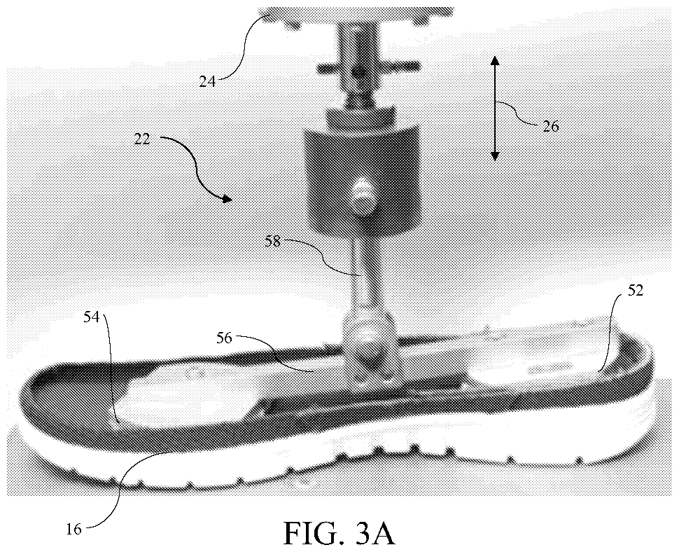

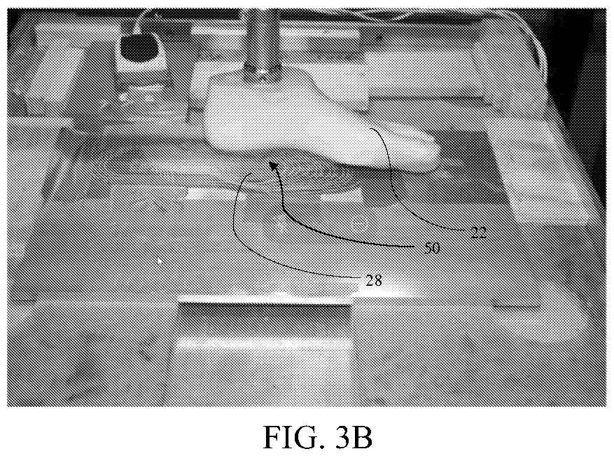

The system 10 can comprise an anvil 30 upon which samples can be positioned. A first sample 32 can comprise the footwear article 14, or a portion thereof, such as, for example, a bottom assembly 16 (shown in FIG. 3A) of the footwear article 14. The bottom assembly 16 can comprise the components of the footwear article that are disposed between the foot of the user and the standing surface, such as, for example, a sole and, optionally, an insert or other foot engagement pad that rests on the sole. In some optional aspects, the first sample can be embodied as the footwear article 14 or the portion thereof. The footwear article 14 can have a first (e.g., lower) surface 34 that rests against the anvil 30 and an opposed second surface 36 that is in engagement with the contact structure 22. In some optional aspects, the footwear article 14 can be, for example, a boot, a dress shoe, a flip-flop, a running shoe, or a high heel shoe. Optionally, the footwear article 14 can be a men's dress shoe or any other appropriate footwear.

A second sample 40 can comprise the footwear article 14 and the flooring material 12 in a stacked arrangement. In some optional aspects, the first sample 32 can consist of the flooring material 12 and the footwear article 14 (or the portion of the footwear article). In some aspects, the footwear article 14 and the flooring material 12 can be stacked along the first axis 26. For example, the flooring material 12 can be positioned between the footwear article 14 and the anvil 30. The flooring material 12 can have a first surface 42 that biases against the anvil 30 and a second, opposing surface 44 that biases against the first surface 34 of the footwear article 14.

Referring also to FIG. 3A, in some aspects, the contact structure 22 can define a contact surface 50. Optionally, the contact surface 50 or contact structure 22 can be representative of a foot. For example, the contact surface 50 can have a heel portion 52 and a ball portion 54 that are positioned for engagement with the footwear article where the foot typically distributes a majority of the pressure to the footwear article 14. In some aspects, the heel and ball portions 52, 54 can be spaced along a transverse axis by an arm 56. The arm 56 can be pivotably coupled to a driver 58 that applies an axial force to the arm along the first axis 26 to allow a force distribution between the heel and ball portions 52, 54 that is representative of the distribution between the heel and ball of the foot of an individual. In various further aspects, the contact structure can comprise a contact surface 50 that is non-pivotably coupled to the driver 58. The contact surface 50 can optionally be a single planar surface or have a complex curvature that simulates at least a portion of a foot. For example, referring to FIG. 3B, the contact structure 22 can be foot-shaped, defining a contact surface 50 that is the shape of a bottom of a foot.

In some aspects, the one or more sensors 28 can comprise a load cell or other force sensor that is configured to determine a contact force between the contact structure 22 and the sample being tested. In further optional aspects, and as illustrated in FIG. 3B, the one or more sensors 28 can comprise a pressure sensor that is configured to determine a plurality of pressures at a plurality of locations across the contact surface 50. In some optional aspects, the one or more sensors 28 can have a sufficient sample detection speed to determine an energy absorption of the samples. In further aspects, the one or more sensors 28 can have a sufficient sample detection speed to determine a peak force during contact between the contact structure and a sample (e.g., over a short duration of an impact that is representative of a heel strike during walking). In some aspects the one or more sensors 28 can comprise a linear displacement sensor that is configured to determine displacement of the contact structure 22 along the first axis 26. It is contemplated that the force data from the force sensor(s) can be correlated with displacement data from the linear displacement sensor. In this way, using numerical integration of the force and displacement data, energy absorbed by the sample can be determined.

In some aspects, one or more sensors 28 can be in communication with the upper surface of the tested sample (e.g., the first sample 32 or the second sample 40). For example, optionally, one or more sensors 28 can be associated with the contact structure 22. In further aspects, one or more sensors 28 can be in communication with the lower surface of the tested sample. For example, optionally, one or more sensors 28 can be associated with the anvil 30.

The system 10 can be used to perform at least a first test on the first sample and at least a second test on the second sample. Each test of the first and second tests can comprise applying a force to the respective sample and, in response, obtaining an output corresponding to a respective comfort indication metric for said respective sample. For example, the system 10 can apply a force to the first sample 32. The force can be applied along the first axis 26. The force to the first sample can be representative of a bodyweight load. For example, the force can be representative of standing bodyweight. In further aspects, the force can be representative of a bodyweight load for an impact due to walking, running, or jumping. Accordingly, the force can be applied as an impulse or in a sustained manner. The force can be applied to the bottom assembly 16 of the footwear article to compress the bottom assembly.

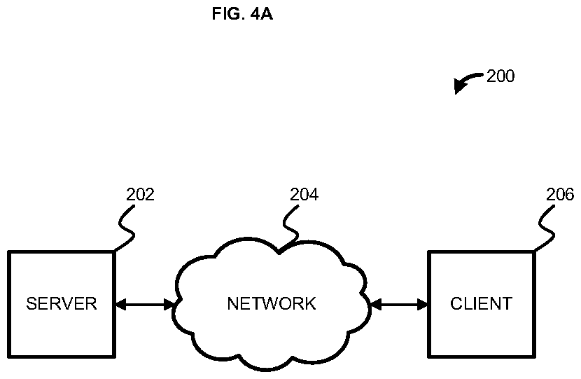

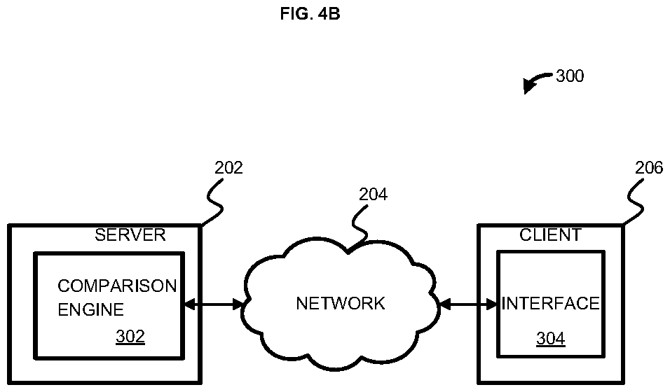

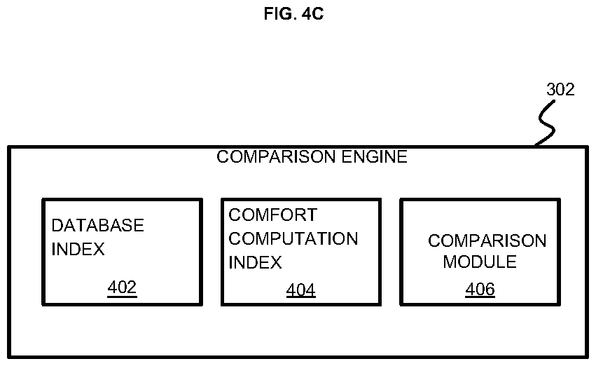

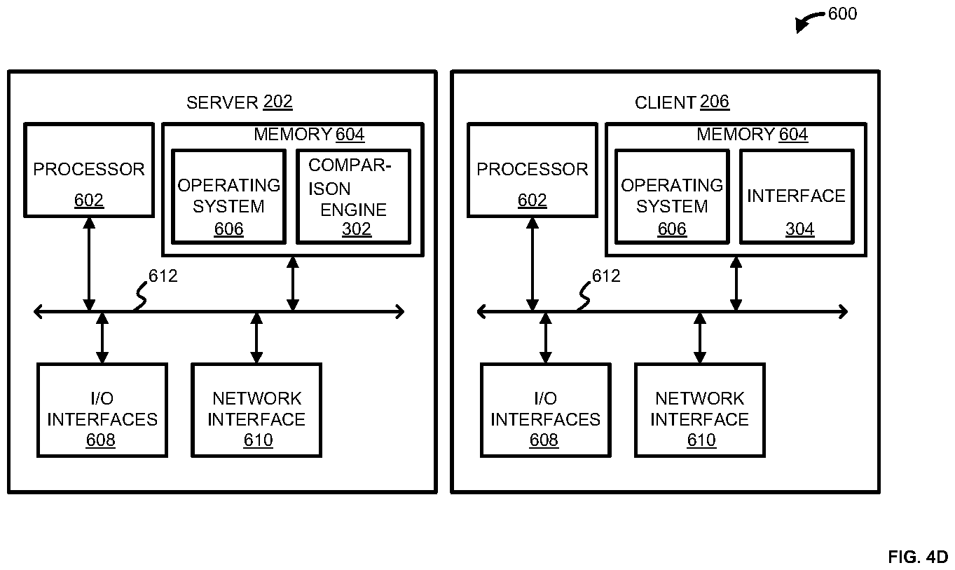

The sensor(s) 28 can generate at least one first output in response to the force applied to the first sample 32. Optionally, the at least one first output can comprise a force, a pressure, and/or an energy value. The at least one first output can be, or be associated with, a comfort indication metric. For example, a comfort indication metric can be a force, a pressure, and/or an energy value output by the sensor(s) 28. In further aspects, the comfort indication metric can be a value calculated based on the outputs of the sensors, such as, for example, energy absorbed as calculated via numerical integration of force and displacement data. For example, it is contemplated that a flooring material comfort characterization device (e.g., operating environment 600 of FIG. 4D), as further disclosed herein, or other computing device can receive one or more distance measurements (e.g., movement of the contact assembly along the first axis 26 from initial contact with the footwear article 14) and at least one force measurement (e.g., a respective force value at each distance measurement) and determine an energy value via numerical integration of the force over the distance traveled. Thus, the energy value can be a function of one or more outputs of a distance sensor and one or more outputs of a force sensor. In further aspects, the energy value can be a calculation based on a measured deceleration.

The system 10 can apply a force to the second sample 40, wherein the force is representative of a bodyweight load. The force can be the same force, in terms of the force applied and the duration of force application, as that which is applied to the first sample 32 to enable relevant comparison. In some optional aspects, the force can be evenly applied to the second sample. Accordingly, the force can be distributed across the sample with an even pressure. In further aspects, the force can be distributed across the sample in a manner representative of force/pressure distributions of a foot. That is, in said further aspects, the force may be distributed unevenly, e.g., more force may be distributed at portions representative of the heel and/or ball of a foot. The force can be applied to the bottom assembly 16 of the footwear article to compress the bottom assembly 16 and the flooring material 12. It is contemplated that the stacked configuration of the bottom assembly 16 of the footwear article 14 and the flooring material can have the effect of distributing the force of the footwear article against the flooring material in a way that is representative of an individual standing on the flooring material while wearing the footwear article. As should be understood, this force distribution can differ from direct application of force from the contact structure 22 against the flooring material. Thus, the disclosed systems and methods solve the technical problem of conveying abstract material measurements by instead capturing measurements that accurately reflect user experience.

The sensor(s) 28 can generate at least one second output in response to the force applied to the second sample 40. The at least one second output can comprise a force, a pressure, and/or an energy value. The at least one second output can be, or be associated with, a comfort indication metric. For example, the comfort indication metric can be a force, a pressure, an acceleration (or deceleration) and/or an energy value output by the sensor(s) 28. In further aspects, the comfort indication metric can be a value calculated based on the outputs of the sensors, such as, for example, energy absorbed as calculated via numerical integration of force and displacement data.

In various aspects, the at least one first output and the at least one second output can comprise respective force displacement graphs. Thus, the area under the curve of the force displacement graphs can be associated with the energy absorbed by the first and second samples.

Accordingly, the at least one first output and at least one second output of the sensor(s) 28 can be indicative of energy absorbed by the first and second samples in response to application of the force to the first and second samples 32, 40. The flooring material comfort characterization device (or other computing device) can be configured to calculate the energy absorbed by (or other comfort indication metric associated with) each of the first or second samples.

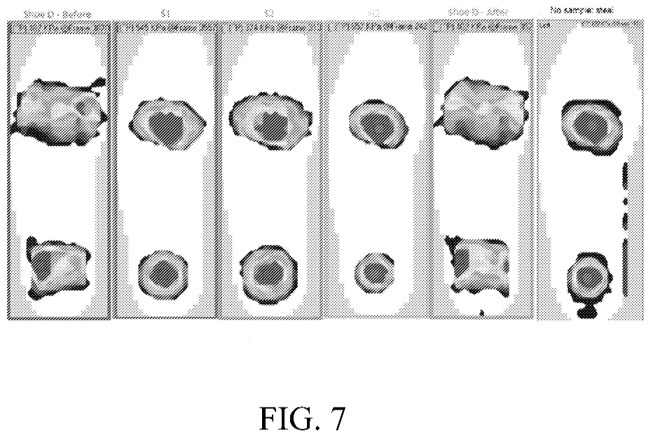

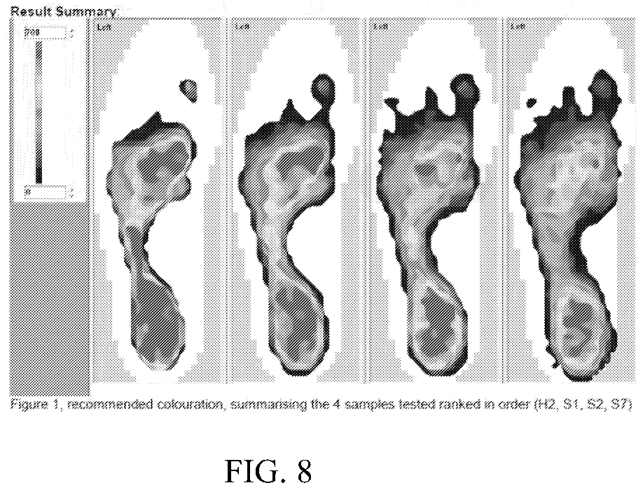

Referring to FIGS. 7 and 8, in further aspects, the comfort indication metric can be, or be associated with, a pressure measurement. FIG. 7 illustrates an exemplary output showing pressure mapping distribution of a test apparatus on different sample surfaces. Peak pressures can be identified. As shown in FIG. 7, from left to right, peak pressures from the exemplary measurements are 487 kPa, 545 kPa, 374 kPa, 657 kPa, 402 kPa, and 667 kPa (with no sample present). As can be seen, different samples can correspond to different peak pressures in different locations and differing pressure distributions when using the test apparatus of FIG. 3A.

FIG. 8 illustrates an exemplary output showing pressure mapping distribution of a test apparatus on different sample surfaces. As can be seen, different samples can correspond to different peak pressures in different locations and differing pressure distributions when using the test apparatus of FIG. 3B. These data can be generated with use of a human foot or a representative structure, thereby providing data most indicative of actual user experience. Optionally, the same samples can be subjected to the test apparatus of FIG. 3A and the test apparatus of FIG. 3B, and respective comfort indication metrics can be generated for each test.

As shown in FIGS. 7 and 8, different samples can each define a peak pressure. Said peak pressure can define the comfort indication metric. In further aspects, the comfort indication metric can be determined by a formula that is a function of pressure and area. For example, it is contemplated that a peak pressure experienced over a first area can be experienced as less comfortable than the same peak pressure experienced over a smaller area. In further aspects, the comfort indication metric can be determined by a formula that is a function of peak pressure and a location on the user's foot (e.g., with different weight given to peak pressure at the heel versus at the toe). Thus, the formula can be defined to generate a comfort indication metric that corresponds to subjective experience of an individual.

A comfort improvement provided by the flooring material in comparison to the footwear article (or portion thereof) can be determined based on the at least one first output and the at least one second output of the sensor(s) 28. The comfort improvement provided by the flooring material 12 can be determined as a difference between a comfort indication metric of the second sample 40 and a comfort indication metric of the first sample 32. For example, a difference between respective comfort indication metrics such, as for example, energy absorbed or peak force measurements, of the first and second samples can be determined. Thus, in some optional aspects, the comfort improvement can be a difference between the energy absorbed by the second sample and the energy absorbed by the first sample.

A relative comfort improvement provided by the flooring material can be determined in comparison to the comfort provided by the footwear article 14 (or portion thereof). For example, the relative comfort improvement provided by the flooring material can be a ratio between the comfort improvement of the flooring material and a comfort indication metric of the footwear article 14 (or portion thereof) or a different footwear article. In some optional aspects, the relative comfort improvement can be, for example, a ratio of (a) a difference between the energy absorbed by the second sample and the energy absorbed by the first sample to (b) the energy absorbed by the first sample. Thus, in one example, a first comfort indication metric provided by a shoe can be 10 units, and a second comfort indication metric provided by a shoe and a flooring material can be 12 units. Thus, the comfort improvement can be 12−10 units, or 2 units. The relative comfort improvement can be calculated as (12−10)/10, or 0.2, or 20%. Accordingly, in some aspects, the relative comfort improvement can be positive, such as for example, an increase in energy absorption. In another example, a first comfort indication metric provided by a shoe can be 10 units, and a second comfort indication metric provided by the shoe and a flooring material can be 8 units. Thus, in this example, the comfort improvement can be 8-10 units, or −2 units. The relative comfort improvement can be calculated as (8−10)/10, or −0.2, or −20%. Accordingly, in some aspects, the relative comfort improvement can be negative, such as for example, a decrease in peak pressure.

In further aspects, a relative comfort provided by the flooring material 12 and footwear article 14 in comparison to the comfort provided by footwear article 14 (or portion thereof) can comprise determining a ratio between (a) at least one output of the sensor(s) 28 from the first output (from testing the first sample) and (b) at least one output of the sensor(s) 28 from the second output (from testing the second sample). Thus, in the example in which the first comfort indication metric provided by the shoe is 10 units and the second comfort indication metric provided by the shoe and the flooring material is 12 units, the relative comfort can be 12/10 or 1.2, indicating a 20% difference between the comfort of the shoe and the comfort of the shoe with the flooring material.

Thus, the disclosed systems and methods solve the technical problem of relating flooring material measurements by comparing measurements associated with experiences with which a lay consumer is familiar.

Referring also to FIG. 4A-D, it is contemplated that a flooring material comfort characterization device (e.g., environment 600), further disclosed herein, can receive a first comfort indication metric associated with the one or more outputs of the sensor(s) 28 in response to application of the force to the first sample and a second comfort indication metric associated with the one or more outputs of the sensor(s) 28 in response to application of the force to the second sample. The flooring material comfort characterization device can relate the first and second comfort indication metrics. For example, the first and second comfort indication metrics can be, respectively, energy absorbed by the first and second samples. In some aspects, the flooring material comfort characterization device can determine a comfort improvement provided by the flooring material (e.g., a difference between the first and second comfort indication metrics). In various aspects, the flooring material comfort characterization device can determine a relative comfort improvement provided by the flooring material. In some aspects, the relative comfort improvement can be the ratio of the difference between the first and second comfort indication metrics to the first comfort indication metric.

It is further contemplated that the relative comfort improvement provided by the flooring material can be compared to a plurality of different shoes and/or a plurality of different shoe types (i.e., to the comfort indication metric of the different shoes or shoe types). In this way, the flooring material can, for example, be compared to the comfort of a shoe (e.g., as comfortable as a dress shoe, as comfortable as an athletic shoe, between the comfort levels of a dress shoe and an athletic shoe, etc.). For example, it is contemplated that the system 10 can be embodied, at least in part, by a conventional system for characterizing shoe comfort. Still further, the method for applying the force and the types of outputs (e.g., force, energy, pressure, linear distance) received from the sensors can be consistent with conventional test methods for characterizing shoe comfort. For example, in some optional aspects, the test system and sample collecting steps of the SATRA TECHNOLOGY Test Method 183 can be used to acquire the test data from the first and second samples. Thus, it is contemplated that the relative comfort improvement provided by the flooring material can be compared to a plurality of comfort indication metrics of different footwear articles. For example, the comfort improvement provided by the flooring material can be related a plurality of shoes and or a plurality of shoe types. Because of the abundance of historical data from testing shoes, it is contemplated that the comfort improvement provided by the flooring material can be compared to comfort indication metrics of shoes not acquired in contemplation of comparison to the flooring material. Accordingly, in some optional aspects, respective relative comfort improvements can be determined as a ratio of the comfort improvement provided by the flooring material to the respective comfort indication metrics of a plurality of different shoes. The respective relative comfort improvements can optionally be determined by the flooring material comfort characterization device as disclosed herein.

In yet further aspects, using the systems and methods disclosed herein, a flooring material can be compared to another flooring material. For example, a first sample can comprise a first flooring material and a footwear article. A second sample can comprise a second flooring material and the footwear article. Each of the first and second samples can provide respective comfort indication metrics. In some aspects, the respective comfort indication metrics can be compared. In further aspects, the comfort indication metric of the footwear article alone can be subtracted from the respective comfort indication metrics of the first and second samples to provide respective comfort improvements associated with the flooring materials, and said respective comfort improvements can be related to each other.

Referring to FIG. 4D, in some optional aspects, the flooring material comfort characterization device (e.g., operating environment 600) can comprise in memory (e.g., memory 604) a database comprising energy absorption properties of a plurality of shoe types. The memory can further comprise instructions that, when executed by the at least one processor, cause the processor(s) (e.g., processor 602) to receive one or more outputs from the at least one sensor in response to application of force to a stacked arrangement of a flooring material and at least a portion of the footwear article. In some aspects, the one or more outputs can be indicative of energy absorbed by the stacked arrangement. The instructions can further cause the processor(s) to determine a shoe type that corresponds to the flooring material and/or determine a relative increase in energy absorption provided by the flooring material in comparison to the shoe type. For example, the processor(s) can calculate respective differences between the energy absorption of a flooring material and energy absorption values from a database comprising energy absorption values for respective shoes/shoe types and output the shoe/shoe type with the lowest calculated difference. As another example, the processor(s) can perform a calculation of the relative increase in energy absorption (as further described herein) and output a calculated value corresponding to the relative increase in energy absorption. Thus, for example, and as further disclosed herein, in some aspects, the flooring material can be associated with a shoe type (or other familiar article, such as another flooring material) of similar comfort. In yet further aspects, the flooring material can be associated with a combination of a footwear article and another flooring material. For example, the comfort provided by a first flooring material (e.g., carpet) without any footwear can be associated with (e.g., compared to) a second flooring material and a footwear article (e.g., an athletic shoe on hardwood).

Data acquired from the methods disclosed herein can be presented to convey comfort provided by the flooring materials as compared to footwear articles or other reference points of known comfort provided to a user (e.g., a yoga mat, a standing desk mat, other flooring material, etc.). For example, a visual aid, such as a bar chart, color scale, or other graph, can show a comfort improvement of the flooring material as compared to the footwear article or a plurality of footwear articles. Optionally, a scale can comprise a plurality of shoes (e.g., high heel, men's work shoe, nursing shoe, tennis shoe) arranged by comfort indication metric. One or more flooring materials can be located along the scale at or between shoes of different comfort indication metrics. In some aspects, the flooring material(s) and shoe(s) can be spaced from each other as a function of their proximity in values. For example, a first flooring material having a similar comfort indication metric to a shoe can be positioned proximate to said shoe than a second flooring material having a more disparate comfort indication metric. In further aspects, a plurality of flooring materials can be evaluated as disclosed herein to determine respective comfort improvements of each. In this way the flooring materials can be compared to each other. For example, the flooring materials can be associated with different categories (e.g., industrial/commercial/residential or high comfort/medium comfort/low comfort). Thus, in some optional aspects, flooring materials can be bracketed into categories delineated by comfort indication metrics (optionally, with each category having its own respective scale/chart/graph). For example, a first chart can comprise a representation (e.g., bar graph) showing a plurality of different comfort indication metrics associated with industrial flooring materials, and a second chart can comprise a representation showing a plurality of different comfort indication metrics associated with commercial flooring materials. Thus, different charts, representations, or comparative data can be provided to a consumer depending on which environment the consumer is seeking to cover with the flooring material. In some optional aspects, a color scale can be associated with the comfort improvements of the flooring materials. For example, red can correspond to low comfort indication metrics, white can correspond to high comfort indication metrics, and pink can correspond to comfort indication metrics between high and low. Optionally, the colors can be provided as gradients in order to convey relative comparison between similar comfort levels.

Still further, it is contemplated that the comfort indication metrics can be used to relate the flooring materials to the standardized shoe comfort curve. The shoe comfort curve can be a shoe comfort curve as is known in the art.

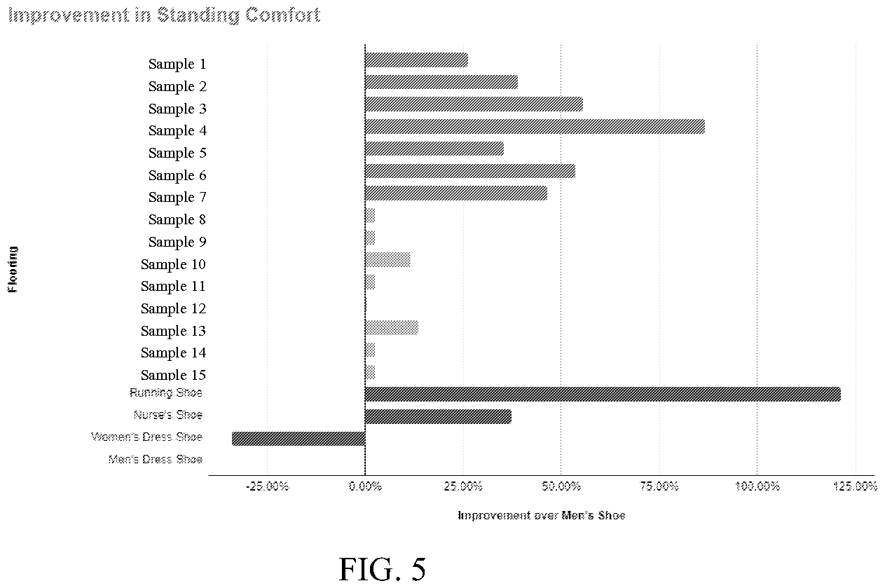

FIG. 5 shows an exemplary bar chart comparing comfort indication metrics of exemplary flooring samples to comfort indication metrics of different shoe types. Each of the samples 1-15 indicates a relative (percent) improvement in comfort indication metric between a men's dress shoe and stack of a men's dress shoe and a different flooring material. Thus, the comfort indication metric of the men's dress shoe presents a baseline from which relative improvement can be measured. The chart below (Table 1) shows approximate relative comfort improvements between a men's shoe and flooring material with the men's shoe as illustrated in FIG. 5. As can be seen, the bar chart shows sample 1 having a relative comfort improvement (between comfort indication metrics) of about 26% and sample 4 having a relative comfort improvement above 80%. This can further be related to a perceived comfort improvement of a user changing from a first shoe type (here, a men's dress shoe) to a second shoe type (that provides improved comfort in comparison to the first shoe type). For example, the comfort indication metric improvement associated with the stack of the men's dress shoe and the flooring material can be related or equated to a change from the men's dress shoe to a second shoe type (that differs from the men's dress shoe). As one representative example, if the stack of the men's dress shoe and the flooring material provides a 35% improvement in comparison to the men's dress shoe alone, then this improvement can be related or equated to a change from a men's dress shoe to a nursing shoe, which can also provide about a 35% improvement. Accordingly, the systems and methods disclosed herein can provide a consumer with tangible, relatable comparisons between comfort of different flooring materials as well as to comfort of different shoes.

TABLE 1

| Sample | Relative Comfort Improvement (%) |
|---|---|
| Sample 1 | 26 |
| Sample 2 | 38 |
| Sample 3 | 55 |
| Sample 4 | 83 |
| Sample 5 | 32 |
| Sample 6 | 54 |
| Sample 7 | 46 |
| Sample 8 | 5 |
| Sample 9 | 5 |
| Running Shoe | 120 |
| Nurse's Shoe | 32 |
| Women's Dress Shoe | −35 |
| Men's Dress Shoe (Baseline) | 0 |

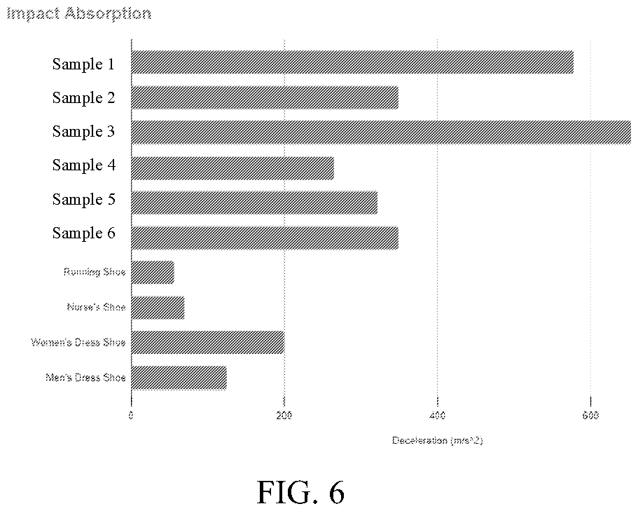

FIG. 6 shows an exemplary bar chart comparing comfort indication metrics of exemplary flooring samples to comfort indication metrics of different shoe types. In particular, samples 1-6 indicate impact absorption measurements for six different sample flooring materials in comparison to different shoe types. In yet further aspects, and as shown in FIGS. 7-8, pressure distributions (e.g., using colors to represent different pressure ranges, as shown) can be displayed. For example, pressure map of a first sample comprising at least a portion of a footwear article and a pressure map of one or more additional samples comprising the portion of the footwear article and a respective flooring material (using the same color or shading scale) can be displayed together to show comfort improvement provided by the flooring sample(s).

The systems and methods disclosed herein can be used to communicate to a consumer that a particular floor product (e.g., a floor product being considered for purchase) provides a similar feel (e.g., standing or walking) to a particular shoe (e.g., a nurse's shoe). For example, within a catalog or other display associated with a particular floor product, comparative data of comfort indication metrics can be provided. Thus, in some aspects, a computing device providing an electronic catalog can provide such comparative data.

In further aspects, a computing device can receive from a user an input corresponding to a particular shoe or shoe type. For example, computing device can provide a prompt to the user, and the user can input, via an interface of the computing device, a particular shoe that the consumer finds comfortable. The computing device can further be configured, based on the input corresponding to the particular shoe or shoe type, provide to the consumer one or more flooring materials that provide a similar comfort indication metric to (e.g., within a predetermined range or a user-selected range) of the particular shoe or shoe type.

Since consumer perception is a personal experience and can vary from one consumer to another, the data may need to be modified and adjusted based on further study or additional parameters such as user feedback. In one example, the flooring material comfort characterization device (e.g., operating environment 600 of FIG. 4D), can be configured to weight or otherwise adjust values in order to most accurately correlate experienced comfort of flooring materials to that of footwear articles, other flooring materials, or any other structure used for comparison. Thus, if a consumer does not like the floor panel or it does not meet the comfort level expectation of the consumer, the consumer can provide feedback to the flooring material comfort characterization device. The feedback can be, for example, a numerical value (e.g., a value corresponding to a consumer-expected comfort indication metric), a subjective metric (e.g., much more than, more than, equal to, less than, or much less than a suggested comfort level), or a recommendation of a different shoe footwear article type, flooring material, or other structure for comparison that more accurately reflects consumer experience. The flooring material comfort characterization device can, for example, apply a weighted scale that is a function of both the consumer feedback and the comfort indication metrics. In some aspects, the flooring material comfort characterization device can provide a customized chart or comparison for the specific consumer. In one example, the flooring material comfort characterization device can add weights to the comfort indication metric based on the consumer feedback. In this way, the flooring material comfort characterization device can tailor its output to a particular consumer.

In further aspects, the flooring material comfort characterization device can use artificial intelligence (e.g., machine learning techniques) to adjust relationships between a particular flooring material and shoe footwear article types, other flooring material(s), or other structure for comparison based on feedback from multiple consumers, crowdsourced information, etc. and/or statistical analysis. For example, the machine learning techniques can process comfort indication metrics (e.g., the first and second outputs, as disclosed herein), consumer feedback from one or a plurality of consumers, and other subjective data to relate flooring materials to shoe footwear article types, other flooring material(s), or other structure for comparison. In this way, if a plurality of consumers, on average, experience a particular flooring material as more comfortable or less comfortable than the comfort indication metrics reflect, the flooring material comfort characterization device can adapt to provide outputs more consistent with user experience.

Accordingly, as shown and described, the disclosed systems and methods solve the technical problem of relating measurements of flooring materials to a lay consumer by comparing the measurements of the flooring material to measurements that the lay consumer can relate to (e.g., measurements corresponding to standing or walking in footwear similar to that worn by the lay consumer). In this way, a consumer can easily identify and relate to differences in comfort of flooring materials. For example, graphical representations or other ways for conveying comfort disclosed herein can guide a consumer to understanding and selecting which flooring material would be preferable. Further, such graphical representations or other ways for conveying comfort can be used to distinguish competitive products.

### Flooring Material Comfort Characterization Device

FIG. 4A and FIG. 4B are block diagrams depicting non-limiting examples of a server 202 and a client 206 connected through a network 204 according to an aspect. The server 202 can comprise one or multiple computers configured to operate a comparison engine 302 (FIG. 4C). The client 206 can comprise one or multiple computers configured to operate an interface 304 such as, for example, a laptop computer, a smartphone, a desktop computer, and the like. Multiple clients 206 can connect to the server 202 through a network 204 such as, for example, the Internet. A user on a client 206 can connect to the comparison engine 302 with the interface 304.

The comparison engine 302 can be configured to receive one or more queries related to a selection of one or more flooring materials or footwear articles. The comparison engine 302 can determine one or more comfort indication metrics associated with the query and make the one or more comfort indication metrics and/or the at least one value comparing said one or more comfort values available for output by the client 206. FIG. 4C is a block diagram depicting an exemplary view of the comparison engine 302 according to an aspect.

The comparison engine 302 can provide one or more outputs indicative of comparisons between comfort indication metrics. In some aspects, said outputs can comprise one or more values. In further aspects, said outputs can comprise one or more representations (e.g., data for generating graphics). The comparison engine 302 can comprise one or more of a database index 402, a comfort computation module 404, and/or a comparison module 406. The database index 402 can comprise a plurality of samples having one or more comfort indication metrics associated therewith. In some aspects, the samples can be footwear articles, flooring materials, or combinations thereof. In further aspects, the database index 402 can comprise a plurality of samples and associated comfort indication metrics from historical data from testing shoes.

The comfort computation module 404 can be configured to receive a comfort indication metric of at least one sample (e.g., a sample comprising a flooring material and a footwear article). The comfort computation module 404 can be configured to provide an output based on the received comfort indication metric. For example, optionally, the comfort computation module 404 can be configured to receive a first comfort indication metric associated with a first sample and a second comfort indication metric associated with a second sample and calculate least one value comparing the first comfort indication metric to the second comfort indication metric (e.g., a comfort improvement, a relative comfort improvement, or a relative comfort). Optionally, the comfort computation module 404 can be configured to receive the first comfort indication metric associated with the first sample and the second comfort indication metric associated with the second sample via user input. Optionally, the comfort computation module 404 can be configured to receive at least one comfort indication metric from the database index 402 and determine a comfort improvement and/or a relative comfort improvement and/or a relative comfort of at least one sample relative to the at least one comfort indication metric from the database index 402.

The comparison module 406 can be configured to generate at least one output comparing at least one sample (e.g., a flooring material sample) to at least one reference (e.g., one or more shoes and/or one or more flooring materials). For example, the comparison module can generate an output that can be converted by a display device to a chart or graph illustrating a comparison of a flooring material sample to at least one reference. In some aspects, the comparison module 406 can receive an output of the comfort computation module 404. For example, the output of the comfort computation module 404 can be represented on a graph with one or more other related comfort indication metrics or values associated with comfort indication metrics (e.g., a comfort improvement, a relative comfort improvement, or a relative comfort).

FIG. 4D is a block diagram depicting an environment 600 comprising non-limiting examples of a server 202 and a client 206 according to an aspect. The server 202 and the client 206 can be a digital computer that, in terms of hardware architecture, generally includes a processor 602, memory system 604, input/output (I/O) interfaces 608, and network interfaces 610. These components (602, 604, 608, and 610) are communicatively coupled via a local interface 612. The local interface 612 can be, for example but not limited to, one or more buses or other wired or wireless connections, as is known in the art. The local interface 612 can have additional elements, which are omitted for simplicity, such as controllers, buffers (caches), drivers, repeaters, and receivers, to enable communications. Further, the local interface may include address, control, and/or data connections to enable appropriate communications among the aforementioned components.

The processor 602 can be a hardware device for executing software, particularly that stored in memory system 604. The processor 602 can be any custom made or commercially available processor, a central processing unit (CPU), an auxiliary processor among several processors associated with the server 202 and the client 206, a semiconductor-based microprocessor (in the form of a microchip or chip set), or generally any device for executing software instructions. When the server 202 or the client 206 is in operation, the processor 602 can be configured to execute software stored within the memory system 604, to communicate data to and from the memory system 604, and to generally control operations of the server 202 and the client 206 pursuant to the software.

The I/O interfaces 608 can be used to receive user input from and/or for providing system output to one or more devices or components. User input can be provided via, for example, a keyboard and/or a mouse. System output can be provided via a display device and a printer (not shown). I/O interfaces 608 can include, for example, a serial port, a parallel port, a Small Computer System Interface (SCSI), an IR interface, an RF interface, and/or a universal serial bus (USB) interface.

The network interface 610 can be used to transmit and receive from an external server 202 or a client 206 on a network 204. The network interface 610 may include, for example, a 10BaseT Ethernet Adaptor, a 100BaseT Ethernet Adaptor, a LAN PHY Ethernet Adaptor, a Token Ring Adaptor, or any other suitable network interface device. The network interface 610 may include address, control, and/or data connections to enable appropriate communications on the network 204.

The memory system 604 can include any one or combination of volatile memory elements (e.g., random access memory (RAM, such as DRAM, SRAM, SDRAM, etc.)) and nonvolatile memory elements (e.g., ROM, hard drive, tape, CDROM, DVDROM, etc.). Moreover, the memory system 604 may incorporate electronic, magnetic, optical, and/or other types of storage media. Note that the memory system 604 can have a distributed architecture, where various components are situated remote from one another, but can be accessed by the processor 602.

The software in memory system 604 may include one or more software programs, each of which comprises an ordered listing of executable instructions for implementing logical functions. In the example of FIG. 4D, the software in the memory system 604 can comprise the comparison engine 302 and a suitable operating system (O/S) 606. In the example of FIG. 4D, the software in the memory system 604 comprises an interface 304 and a suitable operating system (O/S) 606. The operating system 606 essentially controls the execution of other computer programs, such as the comparison engine 302, the interface 304, and provides scheduling, input-output control, file and data management, memory management, and communication control and related services.

The comparison engine 302 can be used for providing an output responsive to an input provided by a user. In an aspect, the input can be one or more keywords, one or more flooring materials or footwear articles, a comfort indication metric, a desired comparison output, combinations thereof, and the like. The comparison engine 302 can use one or more algorithms to store and retrieve relevant search results in a database(s) responsive to the input. For example, the database(s) may index the one or more files according to metadata associated with the one or more files. The comparison engine 302 can comprise an updating algorithm to regularly search for new or updated files. The comparison engine 302 can be configured to operate on one or multiple server(s) 202.

An interface 304 can be used to view web pages on the client 206. The web pages may reside on a network 204 (e.g., Internet) or on a local computer. The interface 304 can be configured to view a web page responsive to an input from a user. The input can be a URL (Uniform Resource Locator) address input directly into the web browser or a hyperlink on a currently viewed web page. Examples of commonly used web browsers include Google Chrome, Microsoft Internet Explorer, Netscape Navigator, and Mozilla Firefox.

In further aspects, the interface 304 can comprise a stand-alone software program. The interface 304 can be configured to receive at least one input from a user. For example, the at least one input can comprise at least one entry of a comfort indication metric. The at least one input can further comprise at least one comparison selection. For example, a comparison selection can be a comparison of a comfort indication metric of a sample to a comfort indication metric to another comfort indication metric stored in the database index 404. In further aspects, the comfort selection can be a comparison to second input comfort indication metric. The interface 304 can further be configured to generate a representation (e.g., a chart or a graph, as further disclosed herein) that is indicative of a comfort indication metric of at least one sample. Optionally, the user interface 304 can enable a user (e.g., by providing a prompt) to select a particular representation that is indicative of the comfort indication metric of the at least one sample.

The comparison engine 302 and/or the interface 304 can be a source program, an executable program (object code), a script, or any other entity comprising a set of instructions to be performed. When the comparison engine 302 and/or the interface 304 is a source program, then the comparison engine 302 and/or the interface 304 can be translated via a compiler, assembler, interpreter, or the like, which may or may not be included within the memory system 604, so as to operate properly in connection with the O/S 606. Furthermore, the comparison engine 302 and/or the interface 304 can be written as (a) an object oriented programming language, which has classes of data and methods, or (b) a procedure programming language, which has routines, subroutines, and/or functions, such as, for example, but not limited to, C, C++, Pascal, Basic, Fortran, Cobol, Perl, and Java.

When the comparison engine 302 and/or the interface 304 is implemented in software, it should be noted that the comparison engine 302 and/or the interface 304 can be stored on any computer readable medium for use by or in connection with any computer related system or method. A computer readable medium can be a non-transitory electronic, magnetic, optical, or other physical device or means that can contain or store a computer program for use by or in connection with a computer related system or method. The comparison engine 302 and/or the interface 304 can be embodied in any computer-readable medium for use by or in connection with an instruction execution system, apparatus, or device, such as a computer-based system, processor-containing system, or other system that can fetch the instructions from the instruction execution system, apparatus, or device and execute the instructions. The computer-readable medium can be any non-transitory means that can store, communicate, propagate, or transport the program for use by or in connection with the instruction execution system, apparatus, or device. The computer readable medium can be, for example but not limited to, an electronic, magnetic, optical, electromagnetic, infrared, or semiconductor system, apparatus, device, or propagation medium. More specific examples (a non-exhaustive list) of the computer-readable medium include the following: an electrical connection (electronic) having one or more wires, a portable computer diskette (magnetic), a random access memory (RAM) (electronic), a read-only memory (ROM) (electronic), an erasable programmable read-only memory (EPROM, EEPROM, or Flash memory) (electronic), an optical fiber (optical), and a portable compact disc read-only memory (CDROM) (optical).

### Exemplary Aspects

In view of the described devices, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: A method comprising:
applying a force to a first sample, the force being representative of a bodyweight load, the first sample comprising a portion of a footwear article;
generating, by at least one sensor, at least one first output in response to application of the force to the first sample, wherein the at least one first output is associated with a first comfort indication metric;
applying the force to a second sample, the second sample comprising a surface covering material positioned beneath the portion of the footwear article in a stacked arrangement;
generating, by the at least one sensor, at least one second output in response to application of the force to the second sample, wherein the at least one second output is associated with a second comfort indication metric; and
determining, based at least in part on the first comfort indication metric and the second comfort indication metric, at least one value comparing the first comfort indication metric to the second comfort indication metric.

Aspect 2: The method of aspect 1, wherein the surface covering material is a floor covering, and wherein determining the at least one value comparing the first comfort indication metric to the second comfort indication metric comprises determining a comfort improvement provided by the surface covering material over the portion of the footwear article.

Aspect 3: The method of aspect 1, wherein the surface covering material is a floor covering, and wherein determining the at least one value comparing the first comfort indication metric to the second comfort indication metric comprises determining a relative comfort improvement of provided by the surface covering material in comparison to the second comfort indication metric of the portion of the footwear article.

Aspect 4: The method of aspect 1, wherein the surface covering material is a floor covering, and wherein determining the at least one value comparing the first comfort indication metric to the second comfort indication metric comprises determining a relative comfort between the first sample and the second sample.

Aspect 5: The method of aspect 2, further comprising: determining, based on the comfort improvement, a relative comfort improvement provided by the flooring material in comparison to the second comfort indication metric of the portion of the footwear article or a third comfort indication metric of a portion of a different footwear article.

Aspect 6: The method of any one of the preceding aspects, wherein the first and second comfort indication metrics comprise respective measurements indicative of energy absorbed by the first and second samples in response to application of the force to the first and second samples.

Aspect 7: The method of aspect 6, further comprising determining, based on the comfort improvement, a relative comfort improvement provided by the surface covering material in comparison to the second comfort indication metric of the portion of the footwear article, wherein the relative comfort improvement is a ratio of a difference between energy absorbed by the second sample and the energy absorbed by the first sample to the energy absorbed by the first sample.

Aspect 8: The method of any one of the preceding aspects, wherein the surface covering material is a floor covering, and wherein the portion of the footwear article comprises a bottom assembly of the footwear article, wherein applying the force to the first sample comprises applying the force to the bottom assembly to compress the bottom assembly, and wherein applying the force to the second sample comprises applying the force to the bottom assembly to compress the bottom assembly and the surface covering material.

Aspect 9: The method of aspect 8, wherein the at least one sensor comprises a force sensor.

Aspect 10: The method of aspect 9, wherein the at least one first output and the at least one second output comprise respective force displacement graphs.

Aspect 11: The method of any one of the preceding aspects, wherein the footwear article is a boot, a dress shoe, a flip-flop, a running shoe, or a high heel shoe.

Aspect 12: The method of aspect 11, wherein the footwear article is a men's dress shoe.

Aspect 13: The method of any one of the preceding aspects, wherein the surface covering material is a floor covering, wherein the force is applied along a first axis, and wherein the surface covering material and the portion of the footwear article of the second sample are stacked along the first axis.

Aspect 14: The method of any one of the preceding aspects, wherein determining the at least one value comparing the first comfort indication metric to the second comfort indication metric comprises: receiving, by a computing device, the first comfort indication metric associated with the at least one first output; receiving, by the computing device, the second comfort indication metric associated with the at least one second output; and determining, by the computing device, a ratio of a difference between the second comfort indication metric and the first comfort indication metric to the first comfort indication metric.

Aspect 15: The method of any one of the preceding aspects, further comprising generating a representation comparing the second comfort indication metric to the first comfort indication metric.

Aspect 16: The method of aspect 15, wherein the surface covering material is a floor covering, and wherein generating the representation comparing the second comfort indication metric to the first comfort indication metric comprises generating a representation comparing a comfort improvement provided by the flooring material to respective comfort indication metrics of a plurality of footwear articles.

Aspect 17: The method of any one of aspects 15 or 16, wherein the surface covering material is a floor covering, wherein generating the representation comparing second comfort indication metric to the first comfort indication metric comprises generating a representation comparing a comfort improvement provided by the flooring material to respective comfort indication metrics of a plurality of other flooring materials.

Aspect 18: The method of any one of aspects 15-17, wherein the representation comparing second comfort indication metric to the first comfort indication metric is one of a graph, a chart, a or color scale.

Aspect 19: The method of any one of aspects 1-15, further comprising generating a representation comparing the second comfort indication metric to respective comfort indication metrics of a plurality of footwear articles.

Aspect 20: The method of any one of aspects 1-16, wherein the portion of the footwear article is an entire footwear article.

Aspect 21: A method comprising:
applying a force to a stacked arrangement of a flooring material and a portion of a footwear article, the force being indicative of a bodyweight load, the flooring material being positioned beneath the portion of the footwear article;
generating, by at least one sensor, at least one output in response to application of the force to the stacked arrangement, wherein the at least one output is indicative of energy absorbed by the stacked arrangement;
determining, by a computing device in communication with a database comprising energy absorption properties of a plurality of shoe types, a shoe type that corresponds to the footwear article; and
determining, by the computing device, a relative increase in energy absorption provided by the flooring material in comparison to the shoe type.

Aspect 22: The method of aspect 21, wherein the portion of the footwear article comprises a bottom assembly of the footwear article, wherein applying the force to the stacked arrangement comprises applying the force to the bottom assembly to compress the bottom assembly and the flooring material.

Aspect 23: The method of aspect 21 or aspect 22, wherein the at least one sensor comprises a pressure sensor.

Aspect 24: The method of any one of aspects 21-23, wherein the at least one output comprises a force displacement graph.

Aspect 25: The method of any one of aspects 21-24, wherein the footwear article is a boot, a dress shoe, a flip-flop, a running shoe, or a high heel shoe.

Aspect 26: The method of aspect 25, wherein the footwear article is a men's dress shoe.

Aspect 27: The method of any one of aspects 21-26, wherein determining, by the computing device, a relative increase in energy absorption provided by the flooring material in comparison to the shoe type comprises determining a ratio between a difference of energy absorbed by the stacked arrangement and energy absorption properties of the shoe type corresponding to the footwear article and energy absorption properties of the shoe type corresponding to the footwear article.

Aspect 28: The method of any one of aspects 21-27, wherein the force is applied along a first axis, and wherein the flooring material and the portion of the footwear article are stacked along the first axis.

Aspect 29: The method of any one of aspects 21-28, further comprising generating, by the computing device, a representation comparing (a) the relative increase in energy absorption provided by the flooring material in comparison to the shoe type to (b) the energy absorption of another shoe type or to a relative increase in energy absorption provided by at least one other flooring material.

Aspect 30: The method of aspect 29, wherein the representation comparing the relative increase in energy absorption provided by the flooring material in comparison to the shoe type to the energy absorption of another shoe type or to the relative increase in energy absorption provided by the at least one other flooring material is one of a graph, a chart, a or color scale.

Aspect 31: The method of any one of aspects 21-30, further comprising generating a representation comparing the relative increase in energy absorption provided by the flooring material to respective energies absorbed by a plurality of footwear articles.

Aspect 32: The method of any one of aspects 21-31, wherein the force is applied by direct contact with the portion of the footwear article.

Aspect 33: The method of any one of aspects 21-32, wherein the portion of the footwear article is an entire footwear article

Aspect 34: A system comprising:
a force application assembly having:
   a contact structure;
   an actuator coupled to the contact structure and configured to effect movement of the contact structure along a first axis; and
   at least one sensor;
at least one processor in communication with the at least one sensor; and
memory in communication with the at least one processor, wherein the memory comprises instructions that, when executed by the at least one processor, cause the at least one processor to:
   receive at least one first output from the at least one sensor in response to application of force to a first sample by the force application assembly, wherein the first sample comprises at least a portion of a footwear article;
   receive at least one second output from the at least one sensor in response to application of force to a second sample by the force application assembly, wherein the second sample comprises a flooring material and the portion of a footwear article provided in a stacked configuration in which the flooring material is beneath the footwear article; and
   determine, based on said at least one first output and said at least one second output, at least one value comparing the at least one first output to the at least one second output.

Aspect 35: A system comprising:
a force application assembly having:
   a contact structure;
   an actuator coupled to the contact structure and configured to effect movement of the contact structure along a first axis; and
   at least one sensor;
at least one processor in communication with the at least one sensor; and
memory in communication with the at least one processor, wherein the memory comprises a database comprising energy absorption properties of a plurality of shoe types, wherein the memory comprises instructions that, when executed by the at least one processor, cause the at least one processor to:
   receive at least one output from the at least one sensor in response to application of force to a stacked arrangement of a flooring material and at least a portion of a footwear article, wherein the at least one output is indicative of energy absorbed by the stacked arrangement;
   determine a shoe type that corresponds to the footwear article; and
   determine a relative increase in energy absorption provided by the flooring material in comparison to the shoe type.

Aspect 36: A method comprising:
applying a force to a first sample, the force being representative of a bodyweight load, the first sample comprising a portion of a footwear article;
generating, by at least one sensor, at least one first output in response to application of the force to the first sample, wherein the at least one first output is associated with a first comfort indication metric;
applying the force to a second sample, the second sample comprising a flooring material positioned beneath the portion of the footwear article in a stacked arrangement;
generating, by the at least one sensor, at least one second output in response to application of the force to the second sample, wherein the at least one second output is associated with a second comfort indication metric; and
associating the first comfort indication metric with the second comfort indication metric.

Aspect 37: The method of aspect 36, wherein associating the first comfort indication metric with the second comfort indication metric comprises determining a ratio of the second comfort indication metric to the first comfort indication metric.

Aspect 38: The method of aspect 36, wherein associating the first comfort indication metric with the second comfort indication metric comprises providing a visual display comparing the second comfort indication metric to the first comfort indication metric.

Aspect 39: The method of aspect 38, wherein the visual display comprises at least one of a graphical display or a numerical display.

Although the foregoing invention has been described in some detail by way of illustration and example for purposes of clarity of understanding, certain changes and modifications may be practiced within the scope of the appended claims.

1. A method comprising:
applying a force to a first sample, the force being representative of a bodyweight load, the first sample comprising a portion of a footwear article;
generating, by at least one sensor, at least one first output in response to application of the force to the first sample, wherein the at least one first output is associated with a first comfort indication metric;
applying the force to a second sample, the second sample comprising a flooring material positioned beneath the portion of the footwear article in a stacked arrangement;
generating, by the at least one sensor, at least one second output in response to application of the force to the second sample, wherein the at least one second output is associated with a second comfort indication metric; and
determining, based at least in part on the first comfort indication metric and the second comfort indication metric, at least one value comparing the first comfort indication metric to the second comfort indication metric.

2. The method of claim 1, wherein determining the at least one value comparing the first comfort indication metric to the second comfort indication metric comprises determining a comfort improvement provided by the flooring material over the portion of the footwear article.

3. The method of claim 1, wherein determining the at least one value comparing the first comfort indication metric to the second comfort indication metric comprises determining a relative comfort improvement of provided by the flooring material in comparison to the second comfort indication metric of the portion of the footwear article.

4. The method of claim 1, wherein determining the at least one value comparing the first comfort indication metric to the second comfort indication metric comprises determining a relative comfort between the first sample and the second sample.

5. (canceled)

6. The method of claim 1, wherein the first and second comfort indication metrics comprise respective measurements indicative of energy absorbed by the first and second samples in response to application of the force to the first and second samples.

7. (canceled)

8. The method of claim 1, wherein the portion of the footwear article comprises a bottom assembly of the footwear article, wherein applying the force to the first sample comprises applying the force to the bottom assembly to compress the bottom assembly, and wherein applying the force to the second sample comprises applying the force to the bottom assembly to compress the bottom assembly and the flooring material.

9. The method of claim 8, wherein the at least one sensor comprises a force sensor, and wherein the at least one first output and the at least one second output comprise respective force displacement graphs.

10. (canceled)

11. The method of claim 1, wherein the footwear article is a boot, a dress shoe, a flip-flop, a running shoe, or a high heel shoe.

12. (canceled)

13. The method of claim 1, wherein the force is applied along a first axis, wherein the flooring material and the portion of the footwear article of the second sample are stacked along the first axis.

14. The method of claim 1, wherein determining the at least one value comparing the first comfort indication metric to the second comfort indication metric comprises:
receiving, by a computing device, the first comfort indication metric associated with the at least one first output;
receiving, by the computing device, the second comfort indication metric associated with the at least one second output; and
determining, by the computing device, a ratio of a difference between the second comfort indication metric and the first comfort indication metric to the first comfort indication metric.

15. The method of claim 1, further comprising generating a representation comparing the second comfort indication metric to the first comfort indication metric.

16. The method of claim 15, wherein generating the representation comparing the second comfort indication metric to the first comfort indication metric comprises generating a representation comparing a comfort improvement provided by the flooring material to respective comfort indication metrics of a plurality of footwear articles.

17. The method of claim 15, wherein generating the representation comparing second comfort indication metric to the first comfort indication metric comprises a representation comparing the comfort improvement provided by the flooring material to respective comfort indication metrics of a plurality of other flooring materials.

18. The method of claim 15, wherein the representation comparing second comfort indication metric to the first comfort indication metric is one of a graph, a chart, a or color scale.

19. The method of claim 1, further comprising generating a representation comparing the second comfort indication metric to respective comfort indication metrics of a plurality of footwear articles.

20. The method of claim 1, wherein the portion of the footwear article is an entire footwear article.

21. A method comprising:
applying a force to a stacked arrangement of a flooring material and a portion of a footwear article, the force being indicative of a bodyweight load, the flooring material being positioned beneath the portion of the footwear article;
generating, by at least one sensor, at least one output in response to application of the force to the stacked arrangement, wherein the at least one output is indicative of energy absorbed by the stacked arrangement;
determining, by a computing device in communication with a database comprising energy absorption properties of a plurality of shoe types, a shoe type that corresponds to the footwear article; and
determining, by the computing device, a relative increase in energy absorption provided by the flooring material in comparison to the shoe type.

22. The method of claim 21, wherein the portion of the footwear article comprises a bottom assembly of the footwear article, wherein applying the force to the stacked arrangement comprises applying the force to the bottom assembly to compress the bottom assembly and the flooring material.

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. A system comprising:
a force application assembly having:
a contact structure;
an actuator coupled to the contact structure and configured to effect movement of the contact structure along a first axis; and
at least one sensor;
at least one processor in communication with the at least one sensor; and
memory in communication with the at least one processor, wherein the memory comprises instructions that, when executed by the at least one processor, cause the at least one processor to:
receive at least one first output from the at least one sensor in response to application of force to a first sample by the force application assembly, wherein the first sample comprises at least a portion of a footwear article;
receive at least one second output from the at least one sensor in response to application of force to a second sample by the force application assembly, wherein the second sample comprises a flooring material and the portion of a footwear article provided in a stacked configuration in which the flooring material is beneath the footwear article; and
determine, based on said at least one first output and said at least one second output, at least one value comparing the at least one first output to the at least one second output.

35. A system comprising:
a force application assembly having:
a contact structure;
an actuator coupled to the contact structure and configured to effect movement of the contact structure along a first axis; and
at least one sensor;
at least one processor in communication with the at least one sensor; and
memory in communication with the at least one processor, wherein the memory comprises a database comprising energy absorption properties of a plurality of shoe types, wherein the memory comprises instructions that, when executed by the at least one processor, cause the at least one processor to:
receive at least one output from the at least one sensor in response to application of force to a stacked arrangement of a flooring material and at least a portion of a footwear article, wherein the at least one output is indicative of energy absorbed by the stacked arrangement;
determine a shoe type that corresponds to the footwear article; and
determine a relative increase in energy absorption provided by the flooring material in comparison to the shoe type.

36. (canceled)

37. (canceled)

38. (canceled)

39. (canceled)
